(19) United States
(12) Patent Application Publication
(10) Pub. No.: US 2023/0374157 A1
(43) Pub. Date: Nov. 23, 2023

(54) SPECIFICY-ENHANCED BISPECIFIC ANTIBODY (SEBA)

(71) Applicants: SYSTIMMUNE, INC., Redmond, WA (US); BAILI-BIO (CHENGDU) PHARMACEUTICAL CO., LTD., Chengdu, Sichuan (CN)

(72) Inventors: Dennis R. GOULET, Redmond, WA (US); Tsung-I TSAI, Redmond, WA (US); Jahan KHALILI, Kirkland, WA (US); Nga Sze Amanda MAK, Mukilteo, WA (US); Hai ZHU, Bothell, WA (US); Yi ZHU, Redmond, WA (US)

(21) Appl. No.: 18/027,569

(22) PCT Filed: Sep. 21, 2021
(86) PCT No.: PCT/US2021/051164

§ 371 Date: Mar. 21, 2023

Related U.S. Application Data
(60) Provisional application No. 63/081,315, filed on Sep. 21, 2020.
(60) Provisional application No. 63/109,877, filed on Nov. 5, 2020.

(51) Int. Cl. C07K 16/46; C12N 15/63; A61P 35/00

(52) U.S. Cl. CPC C07K 16/468; C12N 15/63; A61P 35/00; C07K 2317/31; C07K 2317/35; C07K 2317/55; C07K 2317/52; C07K 2317/622; C07K 2317/92

(57) ABSTRACT

A bispecific tetravalent antibody having a binding specificity to a human epithelium growth factor receptor (EGFR), comprising, from N terminus to C terminus, a Fab region having a first binding specificity to human EGFR, wherein the Fab region comprises a variable region having an amino acid sequence having at least 90% of sequence identity to the sequences as disclosed herein; a Fc domain, and a scFv domain having a second binding specificity to HER3.

### CROSS REFERENCE TO RELATED APPLICATIONS

This application claims the benefit of the filing date of U.S. Provisional Application Ser. No. 63/081,315 filed Sep. 21, 2020, and U.S. Provisional Application Ser. No. 63/109,877 filed Nov. 5, 2020 under 35 U.S.C. 119(e), the entire disclosures of which are incorporated by reference herein.

### TECHNICAL FIELD

The present disclosure generally relates to the technical field of antibody cancer therapeutics, and more particularly relates to bispecific tetravalent antibodies.

### BACKGROUND

The human epidermal growth factor receptor (EGFR, also known as ErbB1, HER1) family has four members, EGFR, HER2, HER3, and HER4. Deregulation of each member by means of mutation, amplification, and overexpression plays an important role in tumorigenesis and tumor metastasis. Overexpression is associated with the development of a wide variety of tumors. Interruption of EGFR signaling, either by blocking EGFR binding sites on the extracellular domain of the receptor or by inhibiting intracellular tyrosine kinase activity, can prevent the growth of EGFR-expressing tumors and improve the patient's condition. For example, HER2 overexpression occurs in 30% of breast cancer patients, indicative of increased disease recurrence and a poor prognosis. Overexpression is also known to occur in stomach, ovarian, and gastric cancer, adenocarcinoma of lung, aggressive forms of uterine cancer, and salivary duct carcinomas. HER2 mutations have been found in non-small-cell lung cancers. The underlying HER2 mutation and amplification produce aberrant growth signals that activate its downstream signaling pathway leading to tumorigenesis. In many of these cases, HER2 dimerizes with HER3 on the surface of tumor cells, which activates PI3K/AKT signalling that promotes tumor growth and survival¹.

Several therapeutic antibodies and small-molecule inhibitors directed against EGFR and HER2 have been approved for use in the treatment of cancer²⁵. Therapeutic anti-EGFR antibodies, such as cetuximab, panitumumab and nimotuzumab, have been approved for treating metastatic colorectal cancer, head and neck squamous cell carcinoma, and glioma^{26,27}. The monoclonal antibodies against either EGFR or HER2 have demonstrated good clinical responses in colon cancer²⁸ squamous cell carcinoma of head and neck²⁹, breast and gastric cancers²⁵.

Trastuzumab (Herceptin) and other agents targeting HER2 have antitumor efficacy in patients with HER2-expressing breast cancer and stomach cancer. Trastuzumab is a monoclonal antibody that binds to HER2 and the binding increases the activity of p27, a protein that halts cell proliferation. Trastuzumab is effective only in cancers where HER2 is overexpressed. One year of Trastuzumab therapy is recommended for all patients with HER2-positive breast cancer who are also receiving chemotherapy, and there is no additional benefit beyond 12 months. Pertuzumab is another monoclonal antibody capable of inhibiting dimerization of HER2 with other receptors, such as HER3, and is a FDA-approved therapeutics for use in combination with trastuzumab and Docetaxel a chemotherapeutic agent for the treatment of metastatic HER2-positive breast cancer^{2,4}.

Despite of these success, the long-term benefit seems to be limited in some patients. Many forms of tumors that initially respond to these therapeutic agents eventually progress due to an acquired resistance to the agents. The development of drug resistance reduces the efficacy of these treatments. In the case of HER2-targeted therapies, the resistance can occur via upregulation of HER3 or its ligand HRG⁵. Therefore, the current therapeutic approaches aiming at inhibiting the activation of HER2/HER3 signalling pathway have failed to provide meaningful clinical benefit^{31,32}.

In summary, monospecific, bispecific, and combination antibody therapies targeting HER2 and/or HER3 currently approved for clinical use have disadvantages of either a low response rate to the treatment or the patient developing resistance to the treatment. There remains a need of a better treatment for these cancers.

### SUMMARY

The present application generally relates to the technical field of antibody therapeutic agents, and more particularly relates to bispecific tetravalent antibodies against members of EGFR family.

In one aspect, the application provides bispecific tetravalent antibodies having a binding specificity to a human EGFR (Epithelium Growth Factor Receptor). In one embodiment, the antibody comprises, from N terminus to C terminus, a Fab region, a Fc domain, and a scFv domain. The Fab region has a first binding specificity to human EGFR. The Fc domain has a second binding specificity to HER3. In one embodiment, the Fab region may include a variable region having an amino acid sequence having at least 70%, 80%, 85%, 90%, 95%, 98%, 99%, or 100% sequence identity to SEQ ID NO. 1 or 3.

In one embodiment, the bispecific tetravalent antibody may include an amino acid sequence having at least 98%, 95%, or 92% of sequence identity to SEQ ID NO. 11, 13, or a combination thereof.

In one embodiment, the first binding affinity may be 2, 3, 4, 5, 6, 7, 8, 9 10, 20, 30, 50, or 100 folds higher than the second binding affinity. In one embodiment, the first binding affinity has a KD less than 20 nM, and the second binding affinity has a KD more than about 50 nM. In one embodiment, the first binding affinity has a KD less than 10 nM, and the second binding affinity has a KD more than about 100 nM. In one embodiment, the first binding affinity has a KD less than 5 nM, and the second binding affinity has a KD more than about 50 nM.

In one embodiment, the first binding affinity has a KD from about 0.1 to about 150 nM, from about 0.5 to about 50 nM, from about 1 to about 10 nM, from about 1 nM to about 25 nM, from about 0.1, 0.5, or 1.0 nM to about 10, 25, or 50 nM. In one embodiment, the first binding affinity has a KD of about 4.61 nM.

In one embodiment, the second binding affinity has a KD from about 10 to about 500 nM, from about 10 to 250 nM, from about 50 to about 250 nM, from about 10 or 50 nM to about 250 or 500 nM. In one embodiment, the second binding affinity has a KD of about 117 nM.

In one embodiment, the Fab region may be stapled with a disulphide bond.

In one embodiment, the tetravalent bispecific antibody may be an isolated monoclonal antibody, a humanized antibody, a chimeric antibody, or a recombinant antibody.

In one embodiment, the bispecific tetravalent antibody includes a human framework region.

In one aspect, the application provides heavy chain, light chain, or a combination thereof. In one embodiment, the heavy chain comprises an amino acid sequence having at least 98% sequence identity to SEQ ID NO. 9, 13, or a combination thereof. In one embodiment, the light chain comprises an amino acid sequence having at least 98% sequence identity to SEQ ID NO. 11.

In one aspect, the application provides CDR sequences has at least 98% sequence identify to the amino acid sequences as disclosed herein.

In one aspect, the application provides isolated nucleic acid encoding the tetravalent bispecific antibody, the light chain, or the heavy chain as disclosed herein.

In one aspect, the application provides expression vector comprising the isolated nucleic acid as disclosed herein. In one embodiment, the expression vector is expressible in a cell.

In one aspect, the application provides host cell comprising the nucleic acid as disclosed herein. In one embodiment, host cell comprising the expression vector as disclosed herein. The host cell may be a prokaryotic cell or a eukaryotic cell.

In one aspect, the application provides methods of producing a tetravalent bispecific antibody, light chain, or heavy chain as disclosed herein. In one embodiment, the application includes the steps of culturing the host cell disclosed herein so that the tetravalent bispecific antibody, light chain, heavy chain is produced.

In one aspect, the application provides immunoconjugate comprising the tetravalent bispecific antibody disclosed herein and a cytotoxic agent. In one embodiment, the cytotoxic agent may be a chemotherapeutic agent, a growth inhibitory agent, a toxin, or a radioactive isotope, or a combination thereof.

In one aspect, the application provides pharmaceutical compositions. In one embodiment, the pharmaceutical composition comprises tetravalent bispecific antibody or immunoconjugates disclosed herein and a pharmaceutically acceptable carrier.

In one embodiment, the pharmaceutical composition comprising radioisotope, radionuclide, a toxin, a therapeutic agent, a chemotherapeutic agent, or a combination thereof.

In one aspect, the application provides methods of treating a subject with a cancer. In one embodiment, the application comprising administering to the subject an effective amount of the tetravalent bispecific antibody or the immunoconjugates as disclosed herein. In one embodiment, the method may further include the step of co-administering an effective amount of a therapeutic agent.

In one embodiment, the therapeutic agent may be an antibody, a chemotherapy agent, an enzyme, or a combination thereof. In one embodiment, the therapeutic agent may include, for example, capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, a derivative or a combination thereof.

In one embodiment, the cancer comprises cells expressing HER3 or EGFR. In one embodiment, the cancer comprises breast cancer, colorectal cancer, pancreatic cancer, head and neck cancer, melanoma, ovarian cancer, prostate cancer, non-small lung cell cancer, small cell lung cancer, glioma, esophageal cancer, nasopharyngeal cancer, kidney cancer, gastric cancer, liver cancer, bladder cancer, cervical cancer, brain cancer, lymphoma, leukaemia, myeloma.

In a further aspect, the application provides solution comprising an effective concentration of the tetravalent bispecific antibody or its immunoconjugates. In one embodiment, the solution is blood plasma in a subject.

In one embodiment, the subject is a mammal. In one embodiment, the subject is a human.

### BRIEF DESCRIPTION OF THE DRAWINGS

The foregoing and other features of this disclosure may become more fully apparent from the following description and appended claims, taken in conjunction with the accompanying drawings. Understanding that these drawings depict only several embodiments arranged in accordance with the disclosure and are, therefore, not to be considered limiting of its scope, the disclosure may be described with additional specificity and detail through use of the accompanying drawings, in which:

FIG. 1 shows the sequence alignments of SI-1X6.4 and SI-71X14 between their heavy chains (A, where all differences are localized to the VH), light chain (B, where all differences are localized to the VK), VH (C), and VK (D);

FIG. 2 depicts binding kinetics (affinity) of bispecific and control antibodies to His-tagged human EGFR extracellular domain using biolayer interferometry sensorgrams;

FIG. 3 shows Biolayer interferometry sensorgrams showing binding kinetics (affinity) to His-tagged human HER3 extracellular domain. Protein IDs are shown at the top of each panel. Note that in contrast to all other measurement, which were determined using AHC sensors, SI-1R12 required setup with AR2G sensors due to lack of Fc domain.

FIG. 4 shows showing binding kinetics (avidity) to biotinylated human EGFR extracellular domain captured onto SA sensors using biolayer interferometry sensorgrams;

FIG. 5 shows the thermal stability of bispecific antibodies using dynamic light scattering (A), and the SEC profile of SI-1X6.4 and SI-71X14, indicating the lower aggregation in SI-71X14 with humanized EGFR binding domain derived from cetuximab (B);

FIG. 6 demonstrates the tandem binding of bispecific antibodies (SI-1X6.4 and SI-71X14) to EGFR, followed by HER3;

FIG. 7 demonstrates the tandem binding of bispecific antibodies (SI-1X6.4 and SI-71X14) to HER3, followed by EGFR;

FIG. 8 shows the surface expression of EGFR family members on Fadu cancer cells;

FIG. 9 shows the potency of SI-1X6.4 and its parental antibody, SI-1C6 and on Fadu cell proliferation;

FIG. 10 shows the potency of SI-71X14 and its parental antibody, SI-71M1, on Fadu cell proliferation;

FIG. 11 shows the potency of SI-1X2 and its parental antibody, SI-1C3, on Fadu cell proliferation;

FIG. 12 shows the potency of SI-1X4.2 and its parental antibody, SI-1C5, on Fadu cell proliferation;

FIG. 13 shows the comparative potency of antibodies, SI-1X6.4, SI-71X14, SI-1C4, and SI-1R12, on Fadu cell proliferation; and

FIG. 14 shows the comparative potency of antibodies, SI-1X6.4, SI-71X14, SI-1C4, SI-71M1, SI-1C6, and SI-1C7, on Fadu cell proliferation.

### DETAILED DESCRIPTION

This disclosure provides bispecific tetravalent antibodies with superior therapeutic properties or efficacies over the currently known anti-EGFR antibodies. In one embodiment, the antibodies target members of EGFR family including, without limitation, EGFR, HER2, and HER3. These bispecific tetravalent antibodies may inhibit different receptor-mediated oncogenic signaling simultaneously therefore overcome resistance in EGFR inhibitor or monoclonal antibody treatment.

The terms “a”, “an” and “the” as used herein are defined to mean “one or more” and include the plural unless the context is inappropriate.

The terms “polypeptide”, “peptide”, and “protein”, as used herein, are interchangeable and are defined to mean a biomolecule composed of amino acids linked by a peptide bond.

The term “antigen” refers to an entity or fragment thereof which can induce an immune response in an organism, particularly an animal, more particularly a mammal including a human. The term includes immunogens and regions thereof responsible for antigenicity or antigenic determinants.

The terms “antigen- or epitope-binding portion or fragment”, “variable region”, “variable region sequence”, or “binding domain” refer to fragments of an antibody that are capable of binding to an antigen (such as EGFR in this application). These fragments may be capable of the antigen-binding function and additional functions of the intact antibody. Examples of binding fragments include, but are not limited to, a single-chain Fv fragment (scFv) consisting of the variable light chain (VL) and variable heavy chain (VH) domains of a single arm of an antibody connected in a single polypeptide chain by a synthetic linker, or a Fab fragment which is a monovalent fragment consisting of the VL, constant light (CL), VH and constant heavy 1 (CH1) domains. Antibody fragments can be even smaller sub-fragments and can consist of domains as small as a single CDR domain, in particular the CDR3 regions from either the VL and/or VH domains³³. Antibody fragments are produced using conventional methods known to those skilled in the art. The antibody fragments can be screened for utility using the same techniques employed with intact antibodies.

The “antigen- or epitope-binding portion or fragment”, “variable region”, “variable region sequence”, or “binding domain” may be derived from an antibody of the present disclosure by a number of art-known techniques. For example, the antigen-binding fragment (Fab) is a region (Fab region) on an antibody that binds to antigens. Purified monoclonal antibodies can be cleaved with an enzyme, such as pepsin, and subjected to HPLC gel filtration. Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen. The appropriate fraction containing Fab fragments can then be collected and concentrated by membrane filtration and the like. For further description of general techniques for the isolation of active fragments of antibodies^{34,35}.

The term “antibody” is used in the broadest sense and specifically covers single monoclonal antibodies and/or recombinant antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, multi-specific or multi-effective, human and humanized antibodies, as well as active fragments thereof. Examples of active fragments of molecules that bind to known antigens include Fab, F(ab′)₂, scFv and Fv fragments, including the products of a Fab immunoglobulin expression library and epitope-binding fragments of any of the antibodies and fragments mentioned above.

The term “Fv” refers to the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

In some embodiments, antibody may include immunoglobulin molecules and immunologically active portions of immunoglobulin molecules, i.e. molecules that contain a binding site and that immunospecifically bind an antigen. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The light chains of antibodies (immunoglobulins) from any vertebrate species can be assigned to one of two clearly distinct types, called kappa and lambda, based on the amino acid sequences of their constant domains. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The subunit structures and three-dimensional configurations of different classes of immunoglobulins are well known.

The term “valency” refers to valency of antibody referring the number of antigenic determinants that an individual antibody molecule can bind. The valency of all antibodies is at least two, whereas “antibody affinity” refers to the tendency of an antibody to bind to a specific epitope at the surface of an antigen, i.e., to the strength of the interaction.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler & Milstein³⁶, or may be made by recombinant DNA methods (see, e.g., U.S. Pat. No. 4,816,567)³⁷. “Recombinant” means the antibodies are generated using recombinant nucleic acid techniques in exogeneous host cells.

Monoclonal antibodies can be produced using various methods, including without limitation, mouse hybridoma, phage display, recombinant DNA, molecular cloning of antibodies directly from primary B cells, and antibody discovery methods^{38, 39, 40}. Monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity^{41,42}.

The term “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art^{43,44}.

The terms “isolated” or “purified” refers to a biological molecule free from at least some of the components with which it naturally occurs. Either “Isolated” or “purified,” when used to describe the various polypeptides disclosed herein, means a polypeptide that has been identified and separated and/or recovered from a cell or cell culture from which it was expressed. Ordinarily, a purified polypeptide will be prepared by at least one purification step. An “isolated” or a “purified” antibody refers to an antibody which is substantially free of other antibodies having different antigenic a binding specificity.

The term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated. While the immunogenic response generally includes both cellular (T cell) and humoral (antibody) arms of the immune response, antibodies directed against therapeutic proteins (anti-drug antibodies, ADA) may consist of IgM, IgG, IgE, and/or IgA isotypes.

The terms “specific binding”, “specifically binds to”, or “is specific for a particular antigen or an epitope” means that the binding is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “affinity” refers to a measure of the attraction between two polypeptides, such as antibody/antigen, receptor/ligand, etc. The intrinsic attraction between two polypeptides can be expressed as the binding affinity equilibrium dissociation constant (KD) of a particular interaction. A KD binding affinity constant can be measured, e.g., by Bio-Layer Interferometry, where KD is the ratio of kdis (the dissociation rate constant) to kon (the association rate constant), as KD=kdis/kon.

Specific binding for a particular antigen or an epitope can be exhibited, for example, by an antibody having a KD for an antigen or epitope of at least about 10-4 M, at least about 10-5 M, at least about 10-6 M, at least about 10-7 M, at least about 10-8 M, at least about 10-9 M, alternatively at least about 10-10 M, at least about 10-11 M, at least about 10-12 M, or greater, where KD refers to the equilibrium dissociation constant of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

It is considered by the application that the bispecific antibody potentially has the advantage over any combination therapy, which often has greater toxicity than a single agent treatment. Bispecific agents, such as bispecific antibody as disclosed in the application, may act as a single agent targeting the same antigens as the combination therapy does but with the increased efficacy and response rate and reduced toxicity when compare to the combination therapy. In comparison to the combination therapy using two monoclonal antibodies, a bispecific antibody therapeutics can be less toxic to patients and/or more potent due to the increased binding specificity.

In one aspect, the application provides a bispecific antibody having a N terminal and a C terminal, comprising at least two binding domains, wherein the binding domain comprises a Fab region and a scFv domain. The scFv domain may be attached to either the N terminal or the C terminal of the antibody. The Fab region and the scFv domain each independently have a binding specificity to different proteins in the EGFR family.

In some embodiments, scFv molecules described herein contain a linker of (GₘS)ₙ that operably links the VH and VL, regardless of the V-region orientation (LH or HL). The remaining positions in the bispecific antibody may be consist of a human IgG Fc or IgG null Fc heavy chain, VH-CH1-Hinge-CH2-CH3, and its corresponding kappa or lambda light chain, VL-CL. Those scFv domains were genetically linked through a linker of (GₘS)ₙ to either N-terminal or C-terminal of IgG heavy chain, resulting in a contiguous˜75 kDa heavy chain monomer peptide. When co-transfected with the appropriate light chain, the final symmetric bispecific molecule may be purified through the human IgG Fc (Protein A) and assayed to assess functional activity.

In one embodiment, the binding domain having the binding specificity to EGFR comprises cetuximab, panitumumab, and nimotuzumab. Cetuximab is an EGFR inhibitor medication used for the treatment of metastatic colorectal cancer and head and neck cancer. Cetuximab is a mouse/human chimeric monoclonal antibody given by intravenous infusion.

In one embodiment, the binding domain having the binding specificity to HER3 comprises MM-111, a bispecific HER2 and HER3 binding protein. MM-111 is a human serum albumin protein (HSA)-backed bispecific antibody fragment comprises one therapeutic binding to HER3, but its binding to HER2 alone is not sufficient to be considered as a therapeutic binding. In contrast, trastuzumab comprises one single therapeutic binding to HER2.

The bispecific antibody disclosed herein has the advantage of recapitulating the synergistic effect of simultaneously binding to both EGFR and HER3 using a single agent. The bispecific tetravalent antibodies may include an immunoglobulin G (IgG) moiety with two heavy chains and two light chains and two scFv moieties being covalently connected to either C or N terminals of the heavy or light chains via a linker, such as (Gly-Gly-Gly-Gly-Ser)ₙ linkers or a (Gly-Gly-Gly-Ser)ₙ linkers or (GₘS)ₙ linkers.

It is known that having a single therapeutic agent poses significant challenges due to the selection of binding moieties and the backbone structure that may affect the binding efficiency in vivo and the therapeutic efficacy in patients. For example, ALM is a bispecific antibody targeting HER2/HER3, which has antiproliferative activity to tumor cells in vitro. But a short circulating half-life makes it an unlikely candidate drug due to rapid renal clearance³.

Both cetuximab and panitumumab are monoclonal antibodies targeting EGFR (Table 1). They differ in their isotypes, i.e. IgG 1 and IgG2, respectively. This implies that the difference in KD values of binding affinity can be beyond the sequences of CDR and FR. Indeed, reformatting a “2-in-1” bivalent bispecific antibody to IgG1 affects the KD values for binding affinity to EGFR and HER3, respectively. SI-1XC6.4 (C3) (WO2016106157A1²⁰, incorporated herein by reference in its entirety, also known as SI-B001 in clinical trials, NCT04603287) is a tetravalent bispecific antibody targeting EGFR and HER3 with improved EC50 when directly compared to that of duligotuzumab (also known as MEHD7945A, “2-in-1” antibody, or SI-1C4 as described in WO2016106157A1²⁰). SI-1X6.4 (C3) comprises the same anti-EGFR binding domain as that of cetuximab and displays differences in affinity KD values (Table 1a). SI-1X6.4 (C3) comprises the same anti-HER3 binding domain as that of MM-111, and their affinity KD values are significantly different from each other (Table 1a). The structural configuration of each bispecific antibody may contribute to differences in the efficacy of killing tumor cells. Since many forms of human cancer overly express either EGFR or HER2 but not HER3, the unforeseen benefit of a reduced affinity KD of the anti-HER3 binding domain may allow SI-B001 to bind to HER3 only on EGFR-positive tumor cells but not on HER3-positive normal cells.

The term, therapeutic binding, is referred to a binding domain that has been tested in clinical trials in the form of antibody therapeutics for safety. The concept of Specificity-Enhanced Bispecific Antibodies (SEBA) defines bispecific antibodies configured to have a combination of therapeutic bindings to two tumor antigens on the same tumor cell but not on normal cells. Using the EGFR family as an example, there are multiple therapeutic binding domains, including those derived from cetuximab, trastuzumab, MM-111, and “2-in-1”, the objective of SEBA is to develop and/or improve bispecific antibodies as a single therapeutic agent comprising therapeutic binding to two members of EGFR family, such as the pairs of EGFR/HER2, EGFR/HER3, or HER2/HER3. Each configuration may reveal different efficacies in the binding specificity, affinity, and avidity, heregulin binding, inhibition of EGFR/HER3 dimerization and downstream signaling, and ultimately, the therapeutic efficacy and cytotoxicity to patients.

A potential shortcoming of cetuximab is that its variable regions were derived from mice. It has been demonstrated that chimeric antibodies retain non-human sequences may have increased capacity for immunogenicity when compared to humanized or human antibodies.⁶ On the other hand, humanization can increase the stability of antibodies by making the framework regions more compatible.⁷ Another concern is the occupied glycan site at VH N85 (Kabat), where Fab glycosylation could affect the biological properties of the antibody, as well as introduce glycan heterogeneity that must be well-controlled during manufacturing.^{8,9} While immunogenicity of cetuximab appears low based on low incidence of anti-cetuximab IgG response (5%), hypersensitivity is a common occurrence due largely to pre-existing IgE antibodies against the galactose-α-1,3-galactose oligosaccharide that modifies the VH when expressed in SP2/0 cells¹⁰. To overcome these liabilities, cetuximab may opt for humanization and removal of post-translational modification sites to stabilize the antibody, and reduce the potential for immunogenicity while retaining high affinity for EGFR. In this context a humanized EGFR binding domain, which has harbored a therapeutic binding domain from cetuximab, may improve therapeutic efficacy of an existing SEBA, SI-B001.

### EXAMPLES

While The following examples are provided byway of illustration only and not byway of limitation. Those of skill in the art will readily recognize a variety of non-critical parameters that could be changed or modified to yield essentially the same or similar results.

### Example 1: SI-71X14, a Bispecific Tetravalent Anti-EGFR×HER3 Antibody

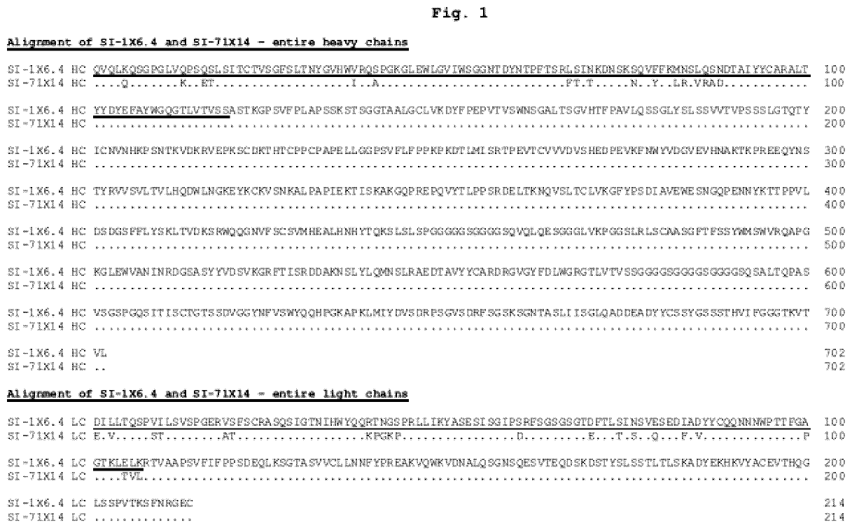

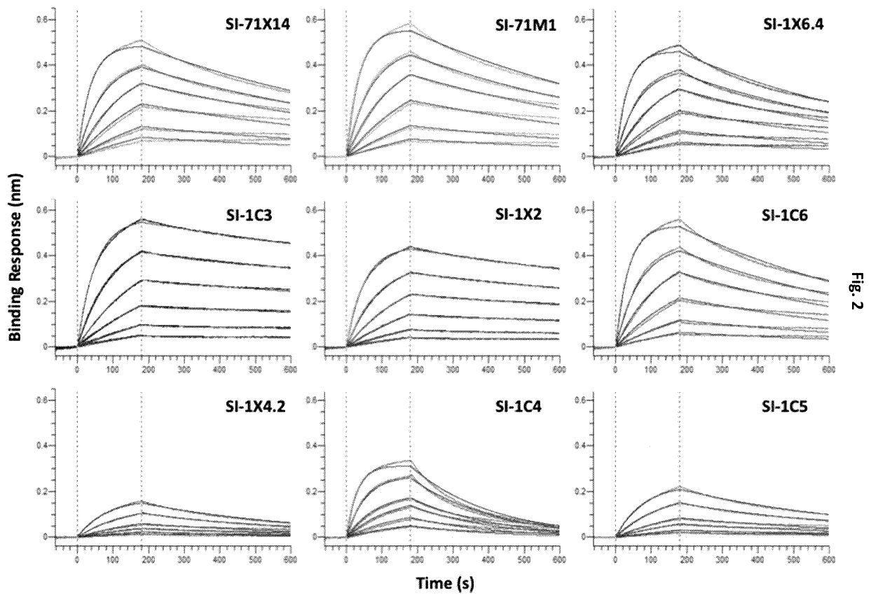

The humanization of cetuximab was designed using different input models with Calculate Mutation Energy set to True (CHARMm forcefield) in order to generate Best Single Mutations sequences. The cetuximab models generated by Discovery Studio's Antibody Modeling Cascade were used. The Input Sequences were cetuximab VH (ending TVSS instead of TVSA) and cetuximab VL. To increase similarity of the VH C-terminus to the consensus sequence in human (FIG. 1), or to make the Vκ C-terminus more Vλ-like, the humanization incorporated changes in the input sequence. After humanization in Discovery Studio, VL was further modified by converting the last three residues of the Vκ domain into their corresponding residues from the λ J-gene. This change was evaluated due to the known importance of the last VL beta strand in determining scFv stability and aggregation propensity, and the more hydrophobic nature of the Vλ terminus, which could provide packing energy to stabilize the interaction^{22, 23,24}. The Top 5 Framework Templates were used with Sequence Similarity Cutoff of 10. CDR loop definition was set to Honegger and Maximum Templates Per Loop was set to 3 with Optimization Level set to High. After generating humanized sequences, VL was further modified by substituting the last four residues of the VL to LTVL to mimic the stable FR4 of lambda antibodies.

Humanized Cetuximab SI-71M1 were designed based on structural analysis of cetuximab, by mutating framework residues to those residues occurring with a frequency of at least 5% in the human germline that caused the most stable structure in silico. Because the energy analysis for this type of humanization depends on the input model, several input structures were examined. SI-71X14 is generated by connecting MM-111's HER3 scFV to C terminal of SI-71M1 heavy chains via (GmS)n linkers.

Thus, SI-71X14 is a modification of SI-1X6.4 where the cetuximab mouse variable regions were replaced with humanized cetuximab variable regions. Except the primary sequences that differ from SI-1X6.4, SI-71X14 is also an αEGFR and αHER3 bispecific tetravalent antibody.

The amino acid changes are shown in FIG. 1. Panel A shows that 17 residue differences in heavy chain sequences are localized to the anti-EGFR cetuximab VH domain. Panel B shows that 22 amino acid differences in the light chain sequences are localized to the anti-EGFR cetuximab VK domain. Panel C zooms in on the VH region to show all amino acid differences in the heavy chains, while panel D zooms in on the VK region to show all differences in the light chains.

In addition to these two bispecific proteins, a number of bispecific and monospecific molecules were included in subsequent assays with properties described in Table 1b. This allowed for comparison of different EGFR and HER3 binding domains as well as different types of structures.

Proteins were expressed by transfecting the expression plasmids for SI-1C7 and SI-1R12 (single plasmid) or co-transfecting heavy and light chains for SI-1C3, SI-1C5, SI-1C6, SI-71M1, SI-1X2, SI-1X6.4, SI-71X14 and SI-1C4, in the ExpiCHO system (Thermo Fisher). Briefly, 10 μg of each expression plasmid (or 20 μg of an unpaired plasmid) was brought to 1 ml with OptiPRO SFM medium. 1 ml of OptiPRO SFM medium containing 80 ul Expifectamine CHO reagent was added to the DNA and incubated at room temperature for 2.5 minutes. The resulting mixture was then added to 25 ml ExpiCHO cells at 6×10⁶ cells/ml in a 125 ml Erlenmeyer flask and incubated at 37° C., 5% CO₂, 150 rpm. Cells were fed with 8.75 ml ExpiCHO feed and 150 μl of CHO enhancer at 24 hours post-transfection and shifted to 32° C., 5% CO₂, 150 rpm. Cells were fed again at 48 hours post-transfection with 8.75 ml ExpiCHO feed. Culture supernatant was harvested 8 days post-transfection, spun for 1 hour at 4500 rpm to pellet the cells and then passed through a 0.2 mm filter.

Fc-containing proteins were purified from the harvested supernatant using a 1-ml MabSelect PrismA protein A column (GE Healthcare). The column was equilibrated with phosphate-buffered saline. The supernatant was then passed through the column at a flow rate of 2 ml/min. The column was washed with 10 ml PBS+0.1% Triton X-100, followed by 10 ml PBS+300 mM NaCl, and finally 10 ml PBS. Protein was then eluted by passing 5 ml of 50 mM sodium acetate, pH 3.5 through the column. The eluted protein was immediately neutralized by addition of 0.5 ml 1M Tris-Cl, pH8.0.

His-tagged scFv proteins were purified from the harvested supernatant using a 1-ml HisTrap HP column (GE). The column was equilibrated with phosphate-buffered saline containing 0.5 M NaCl and 20 mM imidazole, pH 7.4. The supernatant was spiked with 10×binding buffer to reach 0.5 M NaCl and 20 mM imidazole and run over the column at a flow rate of 2 ml/min. The column was washed with 10 column volumes of PBS containing 0.5 M NaCl and 20 mM imidazole, and the protein was eluted using PBS containing 0.5 M NaCl and 500 mM imidazole, pH 7.4.

Immediately after first-step protein A or His tag purification, proteins were analyzed by analytical SEC using Waters Acquity UPLC H-Class with ACQUITY UPLC® Protein BEH SEC 200 Å, 4.6 mm×150 mm, 1.7 μm column. PBS (125 mM sodium phosphate, 137 mM sodium chloride, pH 6.8) was used as mobile phase for 10-minute runs at 0.3 ml/min, injecting 10 μg protein.

Cetuximab have two intrinsic N-glycosylation sites, N85 (Kabat) and N297 (Eu), located in Fab and Fc, respectively. The possible immunogenic N-glycan in the N85 position may impact the pharmacokinetic profile and give rise to anti-drug antibody (ADA). In the humanization version, the position 85 was mutated from N to D, which eliminates the consensus N-glycosylation site, and no glycosylation was detected in any expressed protein. This strategy helped protein purification and characterization but had no effect on the binding affinity as shown in Table 2, 4.

### Example 2: Binding Kinetics to Human EGFR

Monomeric EGFR extracellular domain binding was measured in a biolayer interferometry (BLI) binding assay on an Octet Red 384 instrument (Sartorius). 10 μg/mL of SI-71X14, SI-71M1, SI-1X6.4, SI-1C3, SI-1X2, SI-1C6, SI-1X4.2, SI-1C4, or SI-1C5 was diluted in assay buffer (PBS containing 1% bovine serum albumin and 0.05% Tween 20) and captured on anti-huIgG Fc (AHC) biosensor tips for 180 seconds. Tips were washed for 60 seconds in assay buffer and moved to a human EGFR (expressed and purified in-house) sample in 1:2 serial dilutions from 100 nM to 0 nM. Binding of EGFR extracellular domain to the tips was recorded as biolayer interferometry signals (Δnm) over an association time of 180 seconds. Tips were moved to assay buffer and dissociation was observed for 420 seconds. Sensors were regenerated using 10 mM glycine pH 1.5. Data were globally fit for each antibody to a 1:1 binding model to extract kinetic parameters kₒₙ, k_{dis}, and K_{D} (FIG. 2, Table 2).

Notably, all cetuximab-based proteins had similar binding kinetics to human EGFR. For example, the mAbs SI-1C6 (cetuximab) and SI-71M1 (humanized cetuximab) had K_{D} values of 5.34 and 4.76 nM, respectively. The bispecific (EGFR×HER3) molecules SI-1X6.4 (containing cetuximab variable regions with mouse framework) and SI-71X14 (humanized cetuximab framework) had similar EGFR binding kinetics with K_{D}S of 5.38 and 4.61 nM, respectively. Meanwhile, panitumumab-based mAb (SI-1C3) and bispecific (SI-1X2) proteins had slightly higher affinity with KD values of 2.28 and 2.77 nM, respectively, which was driven by slower dissociation rate. Nimotuzumab-based mAb (SI-1C5) and bispecific (SI-1X4.2) proteins had weaker affinity with KD values of 15.8 and 18.8 nM, respectively, based on slower association kinetics and faster dissociation kinetics. The 2-in-1 bispecific antibody duligotuzumab (SI-1C4) had EGFR affinity of 14.6 nM with the fastest dissociation rate.

### Example 3: Binding Kinetics to Human HER3

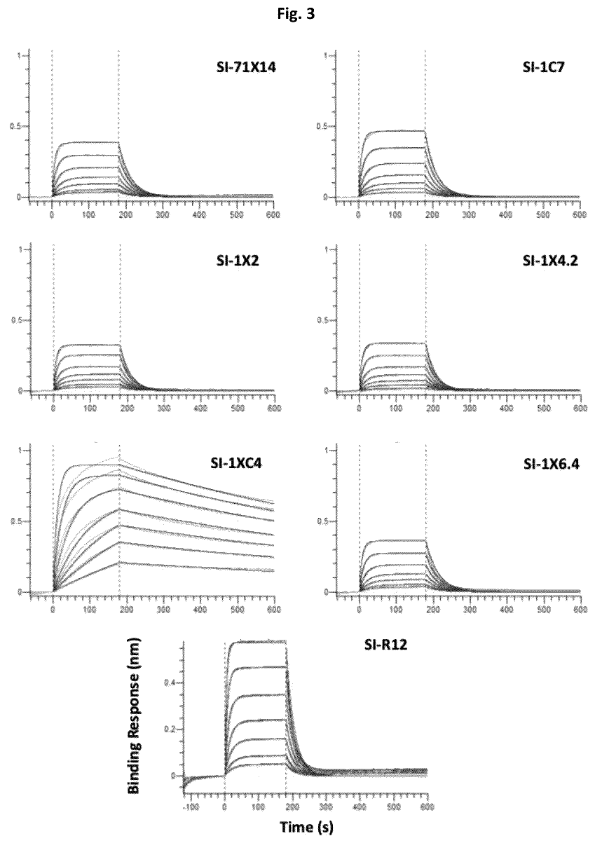

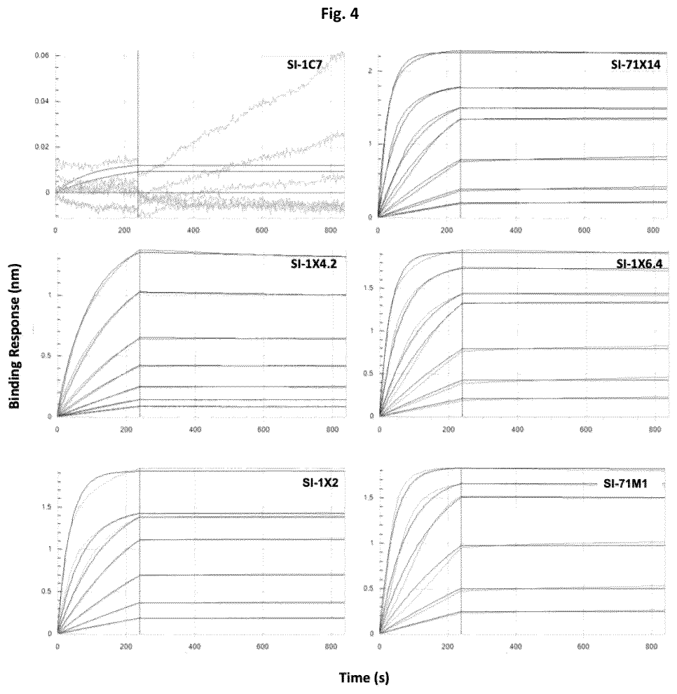

Monomeric HER3 extracellular domain binding was measured in a biolayer interferometry (BLI) binding assay on an Octet Red 384 instrument (Sartorius). 10 μg/mL of SI-71X14, SI-1C7, SI-1C4, SI-1X6.4, SI-1X2, or SI-1X4.2 was diluted in assay buffer (PBS containing 1% bovine serum albumin and 0.05% Tween 20) and captured on anti-huIgG Fc (AHC) biosensor tips for 180 seconds. Tips were washed for 60 seconds in assay buffer and moved to a human HER3 (Acro ER3-H5223) sample in 1:2 serial dilutions from 400 nM to 0 nM. Binding of HER3 extracellular domain to the tips was recorded as biolayer interferometry signals (Δnm) over an association time of 180 seconds. Tips were moved to assay buffer and dissociation was observed for 420 seconds. Sensors were regenerated using 10 mM glycine pH 1.5. Data were globally fit for each antibody to a 1:1 binding model to extract kinetic parameters kₒₙ, k_{dis}, and K_{D} (FIG. 3, Table 3).

Notably, all proteins whose anti-HER3 domain was derived from MM-111 had similar binding kinetics to human HER3. For example, cetuximab-based bispecific proteins SI-1X6.4 (cetuximab variable regions with mouse framework) and SI-71X14 (humanized cetuximab framework) had K_{D} values of 107 and 117 nM, respectively. Panitumumab- and nimotuzumab-based bispecific antibodies SI-1X2 and SI-1X4.2 had HER3 K_{D} values of 131 and 146 nM, respectively. Finally, a control Fc-scFv protein with the same anti-HER3 domain (SI-1C7) had similar binding kinetics with K_{D} of 149 nM. The 2-in-1 bispecific antibody duligotuzumab (SI-1C4), which has distinct anti-HER3 variable regions from the other bispecific proteins, had significantly tighter HER3 binding with K_{D} 4.29 nM.

Due to lack of Fc domain, another comparator bispecific protein (SI-1R12=MM-111, HER2×HER3 albumin fusion) was tested in a different assay format on the same Octet instrument using AR2G sensors. 20 μg/mL of SI-1R12 was diluted in 10 mM acetate pH 6.0 and covalently coupled using EDC/NHS according to manufacturer's instructions using a 600-second loading step. After immobilization of SI-1R12, tips were washed for 120 seconds in assay buffer and moved to a human HER3 (Acro ER3-H5223) sample in 1:2 serial dilutions from 400 nM to 0 nM. Binding of HER3 extracellular domain to the tips was recorded as biolayer interferometry signals (Δnm) over an association time of 180 seconds. Tips were moved to assay buffer and dissociation was observed for 420 seconds. Data were globally fit for each antibody to a 1:1 binding model to extract kinetic parameters kₒₙ, k_{dis}, and K_{D} (FIG. 3, Table 3). Kinetics of SI-1R12 binding to HER3 were similar to that of other bispecific proteins tested, with a K_{D} value of 95.6 nM.

### Example 4: Simultaneous Binding to Human EGFR and HER3

Bispecific binding to EGFR and HER3 extracellular domains was measured in a sandwich-type biolayer interferometry (BLI) binding assay on an Octet RED 384 instrument (Sartorius). After a 180-second baseline step in assay buffer (PBS with 1% BSA and 0.05% Tween 20), biotinylated human EGFR (Acro EGF-H82E3) was loaded onto SA sensors at 5 μg/ml in assay buffer for 240 seconds. Following another 180-second baseline step, association with 2-fold serial dilutions (0 to 100 nM) of SI-1C7, SI-1X2, SI-1X4.2, SI-1X6.4, SI-71M1, or SI-71X14 in assay buffer was performed for 240 seconds followed by a 600-second dissociation in assay buffer without any protein. Immediately following this antibody-binding step, another association step with 500 nM HER3 ECD (in-house expressed) was performed, followed by a 600-second dissociation phase. Each association/dissociation event were separately fit using a 1:1 binding model to extract the binding kinetics for bispecific EGFR and HER3 binding.

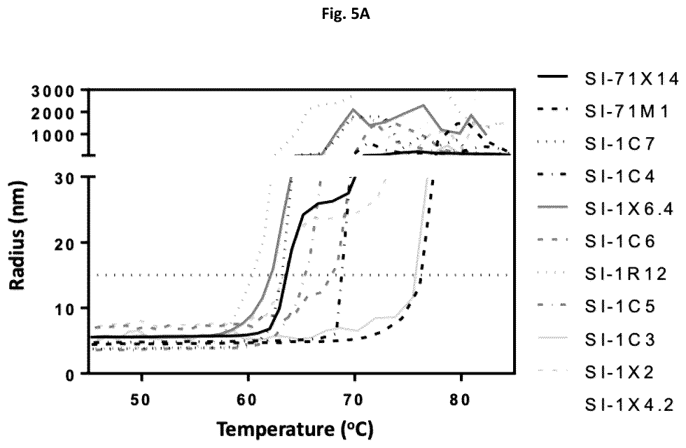

The first binding event measured in this assay is that of antibodies binding to immobilized EGFR, which represents the avidity of the interaction as it might occur on the cell surface. These data are shown in FIG. 4 and kinetic parameters are shown in Table 4. The data demonstrate that cetuximab-based antibodies including humanized cetuximab mAb (SI-71M1) and bispecific cetuximab x anti-HER3 antibodies SI-1X6.4 (cetuximab variable regions with mouse framework) and SI-71X14 (humanized cetuximab framework) all had very high avidity to immobilized EGFR where the K_{D} of the interaction was too tight to be accurately quantified but estimated as less than 1 pM. This high avidity was driven by very slow dissociation rate. Similarly, the panitumumab-based EGFR×HER3 bispecific antibody SI-1X2 also had high avidity with estimated K_{D} less than 1 pM. Nimotuzumab-based EGFR×HER3 bispecific antibody SI-1X4.2 also had strong avidity with fitted K_{D} value of 398 pM. As expected, the Fc-scFv protein specific for HER3, SI-1C7, did not show binding to EGFR.

The second event of interest is the captured antibody (already bound via its anti-EGFR domains) binding to HER3 protein in solution. The kinetic parameters for these interactions are tabulated in Table 5. Cetuximab-based EGFR×HER3 bispecific antibodies SI-1X6.4 (cetuximab variable regions with mouse framework) and SI-71X14 (humanized cetuximab framework) had similar HER3 K_{D} values of 617 and 922 nM, respectively. Panitumumab- and nimotuzumab-based EGFR×HER3 bispecific antibodies SI-1X2 and SI-1X4.2 had similar HER3 affinities of 770 and 165 nM, respectively. Notably, humanized cetuximab mAb (SI-71M1) did not show binding in this assay step due to lack of specificity for HER3, while Fc-scFv protein SI-1C7 targeting HER3 showed no binding response due to lack of loading during the EGFR binding step. Thus, the sandwich assay demonstrates that EGFR×HER3 bispecific antibodies are able to simultaneously bind EGFR and HER3, while proteins with only specificity to either EGFR or HER3 did not show a response in the assay.

### Example 5: Improved Thermal Stability

Dynamic light scattering on a Wyatt DynaPro Plate Reader III was performed for protein thermal stability analysis. Proteins were diluted to 1 mg/ml in 25 mM sodium acetate, 75 mM sodium chloride, 5% (w/v) sucrose, pH 5.5 in 30 μl/well. Temperature was ramped from 25° C. to 85° C. at 1.0° C./min while monitoring the radius. Due to difficulty fitting the differently shaped unfolding curves reproducibly, the temperature at which the radius surpassed 15 nm was used as an objective metric of thermal stability. Samples were run in duplicate, and buffer alone was run as negative control.

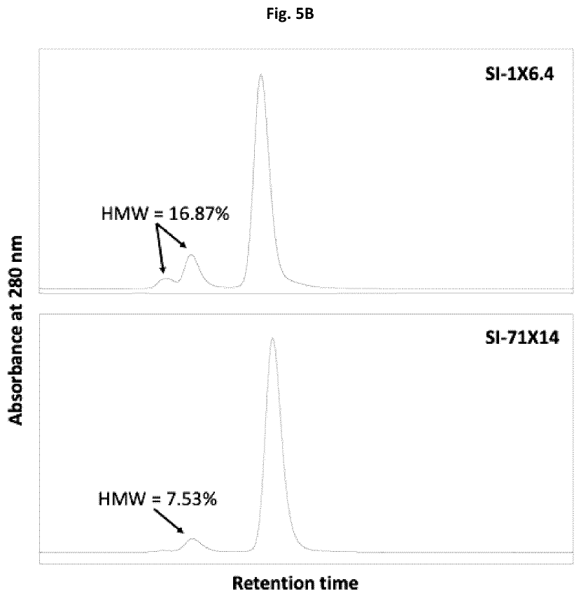

FIG. 5 shows the thermal melting curves of SI-71X14, SI-71M1, SI-1C7, SI-1C4, SI-1X6.4, SI-1C6, SI-1R12, SI-1C5, SI-1C3, SI-1X2, and SI-1X4.2 and Table 6 shows Tm values for these proteins. EGFR mAbs panitumumab (SI-1C3), nimotuzumab (SI-1C5), cetuximab (SI-1C6), and humanized cetuximab (SI-71M1) had Tm values of 77.05, 65.79, 68.39, and 77.80° C., respectively. Thus, humanization of cetuximab not only increased thermal stability by >9° C., but also generated the most stable EGFR mAb of the four tested. Bispecific EGFR×HER3 antibodies based on panitumumab (SI-1X2), nimotuzumab (SI-1X4.2), cetuximab (SI-1X6.4), and humanized cetuximab (SI-71X14) had Tm values of 63.73, 63.79, 62.33, and 64.10° C., respectively. Thus, addition of anti-HER3 scFv to the C-terminus of EGFR mAbs tended to normalize and decrease thermal stability. Notably, the bispecific EGFR×HER3 antibody based on humanized cetuximab had the highest thermal stability of this panel, and increased stability of the parent cetuximab protein by 1.77° C. The control protein based on antibody Fc fused to anti-HER3 scFv (SI-1C7) had Tm of 63.17° C., which is similar to that of bispecific molecules containing this anti-HER3 scFv domain. The bispecific antibody SI-1C4 had Tm of 69.80° C., confirming the high stability of the mAb-like platform. Finally, the MM-111 bispecific HSA fusion (SI-1R12) had the lowest thermal stability with a Tm of 60.41° C. This result demonstrates the favorable stability of the antibody format compared to other protein scaffolds.

### Example 6: Sequential Binding to Human EGFR and HER3

Bispecific binding to EGFR and HER3 extracellular domains was measured in a tandem biolayer interferometry (BLI) binding assay on an Octet RED 384 instrument (Sartorius).

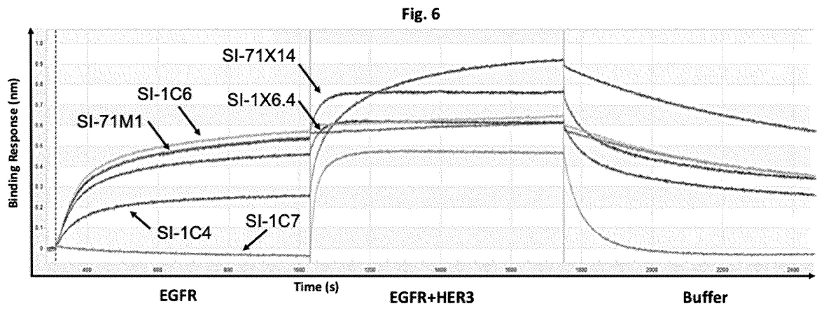

In one format, antibody protein was captured onto AHC sensors, followed by a first association step with EGFR, followed by a second association step with HER3, followed by a dissociation step (FIG. 6). In particular, after a 20-second baseline step in assay buffer (PBS with 1% BSA and 0.05% Tween 20), 10 μg/ml of antibody protein was loaded for 180 seconds, followed by a 60-second baseline step. Next, the first association step with 100 nM of EGFR (purified in-house) was performed for 720 seconds, followed by the second association step with 100 nM of EGFR and 400 nM of HER3 (Acro ER3-H5223) for 720 seconds. Of note, the second step contained HER3 protein, but additionally contained the same amount of EGFR as in the first step (100 nM), so that dissociation of EGFR would not complicate the kinetics observed in the second step. Finally, a 720-second dissociation step was performed.

In the assay with tandem EGFR then HER3 steps, the control EGFR mAbs SI-1C6 and SI-71M1 showed a large response in the EGFR phase, with no significant increase in response during the HER3 phase, indicating binding to EGFR in the first step, but not HER3 in the second step. The control Fc-scFv protein targeting HER3, SI-1C7, showed no binding during the first EGFR step, but a large response during the second HER3 step. The 2-in-1 control mAb SI-1C4 showed binding during the EGFR and HER3 steps, indicating that the first EGFR step was not sufficient to saturate antibody binding so that additional molecules of HER3 could bind in the second step. Bispecific EGFR×HER3 antibodies SI-1X6.4 and SI-71X14 showed significant binding response during both EGFR and HER3 steps, confirming that these proteins are able to simultaneously bind to molecules of EGFR and HER3.

In the assay, we also observe SI-71X14 have better binding response than SI-1X6.4 (nm in Y axis), about 0.1 nm, for EGFR and EGFR/HER3 association step, it implies the binding quantity of SI-71X14 is higher than SI-1X6.4.

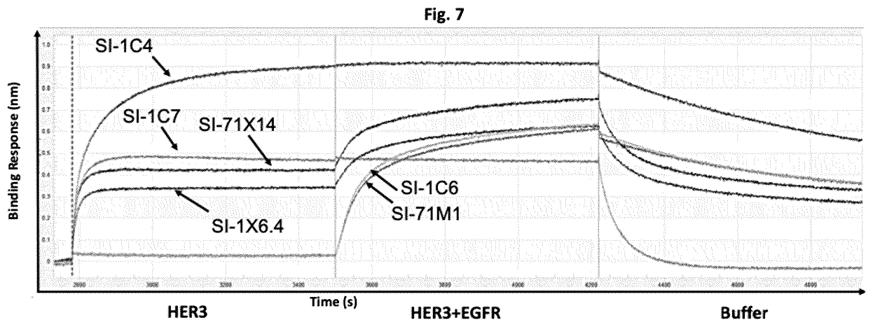

In another format, antibody protein was captured onto AHC sensors, followed by a first association step with HER3, followed by a second association step with EGFR, followed by a dissociation step (FIG. 7). In particular, after a 20-second baseline step in assay buffer (PBS with 1% BSA and 0.05% Tween 20), 10 μg/ml of antibody protein was loaded for 180 seconds, followed by a 60-second baseline step. Next, the first association step with 400 nM of HER3 (Acro ER3-H5223) was performed for 720 seconds, followed by the second association step with 100 nM of EGFR (purified in-house) and 400 nM of HER3 for 720 seconds. Of note, the second step contained EGFR protein, but additionally contained the same amount of HER3 as in the first step (400 nM), so that dissociation of HER3 would not complicate the kinetics observed in the second step. Finally, a 720-second dissociation step was performed.

In the assay with tandem HER3 then EGFR steps, the control EGFR mAbs SI-1C6 and SI-71M1 showed no response during the HER3 binding step followed by large response during the EGFR step, as expected. The control Fc-scFv protein targeting HER3, SI-1C7, showed binding during the first HER3 step, but no binding during the second EGFR step, as expected. The 2-in-1 control mAb SI-1C4 showed significant binding during the first HER3 step, but no additional binding during the second EGFR binding step. The interpretation is that both of this antibody's Fab regions bound to HER3 during the first step, such that there was no free Fab to bind to EGFR during the second step. Bispecific EGFR×HER3 antibodies SI-1X6.4 and SI-71X14 showed significant binding response during both EGFR and HER3 steps, confirming that these molecules are able to simultaneously bind to molecules of EGFR and HER3. Importantly, the tetravalent nature of the SI-1X6.4 and SI-71X14 structure, along with separate binding domains for each antigen, allowed these antibodies to bind concurrently to EGFR and HER3 where this phenomenon was not possible for the 2-in-1 control mAb SI-1C4.

In the assay, we also observe SI-71X14 have better binding response than SI-1X6.4 (nm in Y axis), about 0.1 nm, for HER3 and EGFR/HER3 association step, it implies the binding quantity of SI-71X14 is higher than SI-1X6.4.

Taken together, the characterization of binding kinetics implies a mode of action for SEBA, i.e. specificity enhanced bispecific antibodies, such as SI-71X14 and SI-1X6.4. Unlike the in vitro kinetics, the response and effectiveness of the bispecific antibody treatment may depend on tissue distribution of EGFR and HER3. In patients with various forms of solid tumors, the expression of EGFR may be deregulated by tumor cells while HER3 may be expressed by both normal and tumor cells. Incidentally, many anti-HER3 antibody therapies have failed due to the safety issue, indicating that targeting normal cells may have outweighed the tumor cells in vivo. In this application, the result shows that both SI-1X6.4 and SI-71X14 can enact a sequential binding mode and that SI-71X14 with a humanized EGFR binding domain unveils an improved binding kinetics. The differentiated KD value between EGFR (strong) and HER3 (weak) underlies the selective binding of both SI-71X14 and SI-1X6.4 in favor of binding to EGFR-expressing cancer cells relative to HER3 positive normal cells. In this context, SEBA may help achieve reduced side effects in vivo. Furthermore, having differentiated binding affinity to two tumor-associated antigens (TAA), as measured by strong and weak KD values, may be a new strategy for designing SEBA to target cancer-causing receptors.

### Example 7: Inhibiting Tumor Cell Proliferative

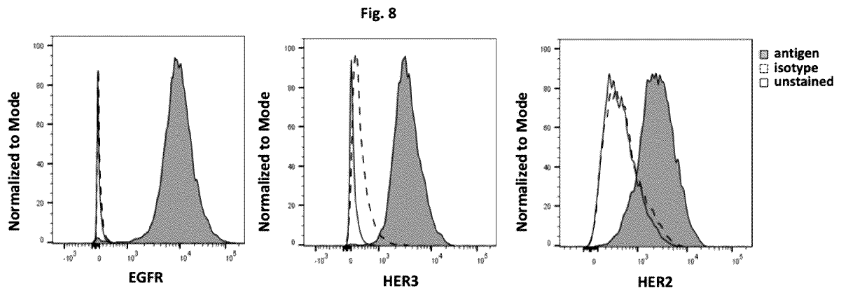

To assess the growth inhibitory potential of anti-EGFR domain containing antibodies, the effect of the cetuximab-derived EGFR domain (wt) and the humanized EGFR binding domain were compared while in different therapeutic formats. The growth inhibitory effects were tested against the Fadu cell line (hypopharyngeal squamous cell carcinoma, ATCC HTB-43) which expresses both EGFR and HER3 proteins, as well as HER2 protein (FIG. 8). Specific antigen presentation was determined by incubation of Fadu cells with fluorescent conjugated antibodies specific for either EGFR, HER3, or HER2 and isotype match control antibodies. Antibody binding was quantified on cells using FACS method (BD Bioscience LSR-Fortessa).

The cell line was seeded in 96-well tissue culture plates at a density of 5000 cells per well in 200 uls of RPMI-1640 medium containing 1% FBS. Treatments were added within the dose range of 90 nM to 85.8 fM. Cells were cultured in the presence of the test antibodies for 63 hours in triplicate plates. Nuclei counts were obtained based on the Fadu cell line stable expression of nuclear localized fluorescence reporter protein mKate2 using time-series microscopy (Incucyte Zoom). Data was collected at baseline, and at intervals during culture. Normalized proliferation is reported based on well seeding and untreated control conditions. Comparative effects of the wt cetuximab and the humanized cetuximab anti-proliferative effect were represented in dose response curves and the IC-50 of inhibition provided based on regression analysis using Sigmoidal, 4PL, Least squares fitting, where X is concentration, and curve fit is provided as R² value in figures (Graphpad Prism 9).

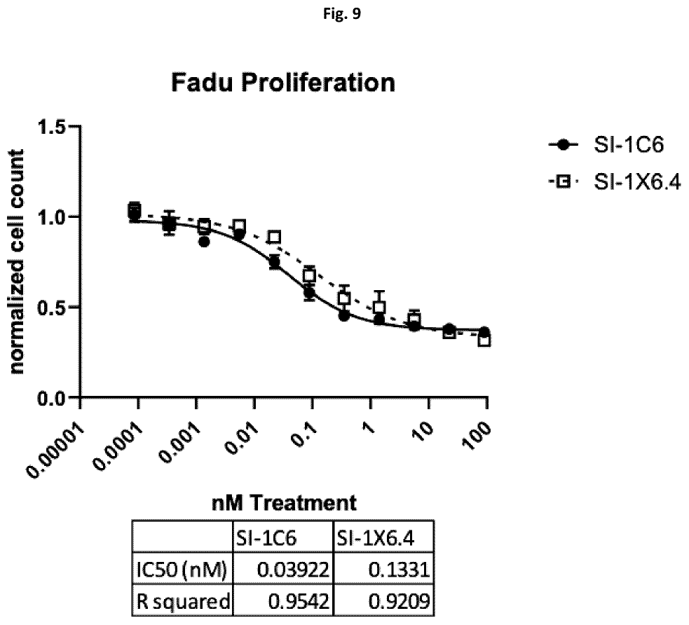

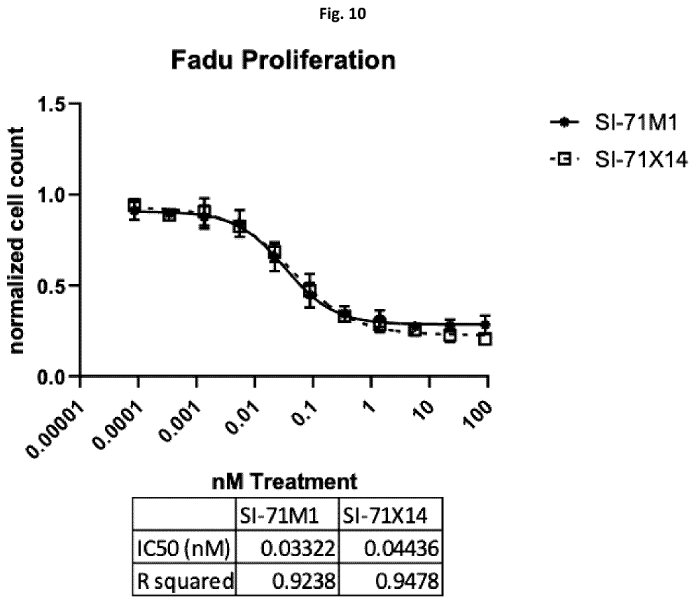

When the wt cetuximab domain is utilized in the bi-specific format with HER3 (SI-X6.4), the IC-50 is reduced 3 fold compared to the EGFR mAbs alone (SI-1C6), while the addition of the HER3 domain provides a greater overall anti-proliferative effect (FIG. 9). However, the humanized cetuximab domain restores the anti-proliferative IC-50 when combined with HER3 binding domain (SI-71X14) in comparison to the humanized cetuximab mAb alone (SI-71M1), and retains the greater overall anti-proliferative effects at higher concentrations (FIG. 10).

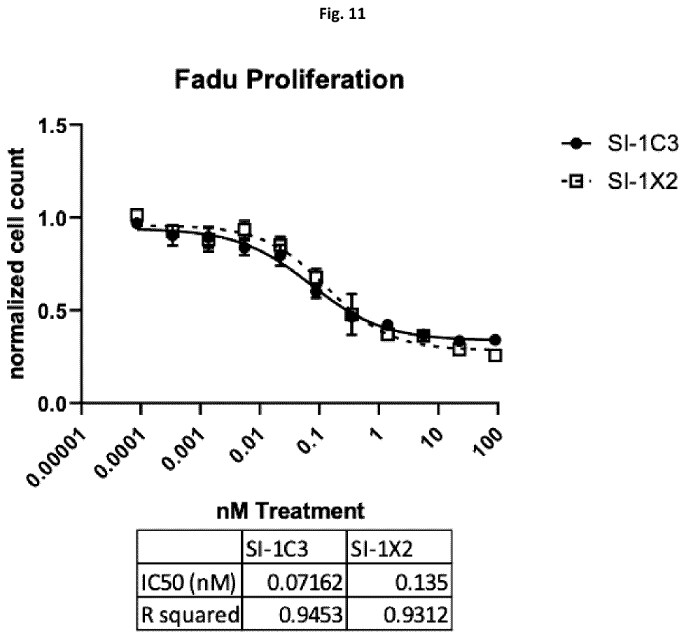

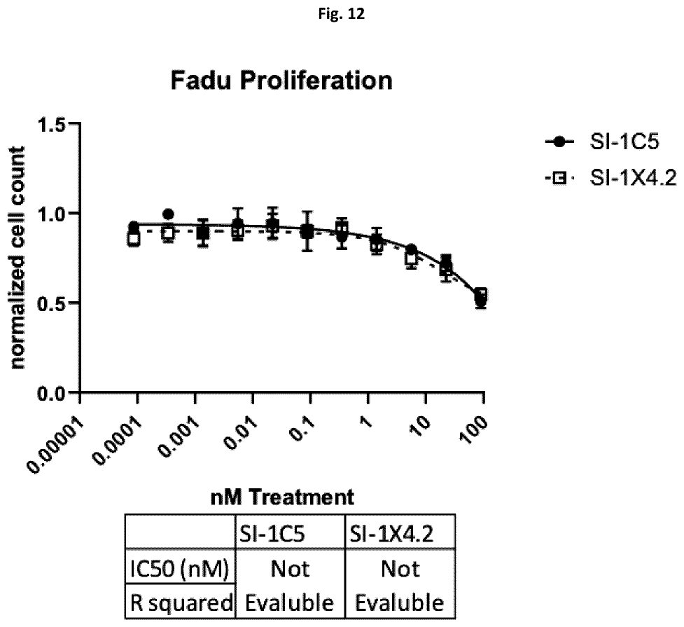

Consistent with the role of HER3 enhancing the function of EGFR inhibition in the bi-specific format, panitumumab (SI-1C3) when engineered in the bi-specific formation with HER3 domain (SI-1X2) achieves a greater overall anti-proliferative effect (FIG. 11). Whereas, the EGFR antibody nimotuzumab (SI-1C5) shows poor capability to inhibit Fadu cell proliferation in this assay system, and the addition of HER3 to nimotuzumab in the bi-specific formation (SI-1X4.2) is not observed to provide a benefit, attesting to the key requirement of the EGFR domain facilitating the anti-proliferative benefit to enable the benefit of HER3 blocking in the bispecific drug design (FIG. 12).

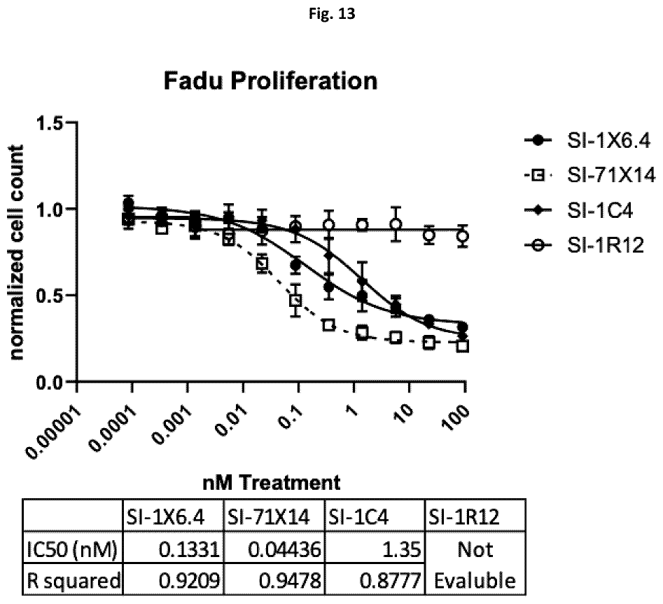

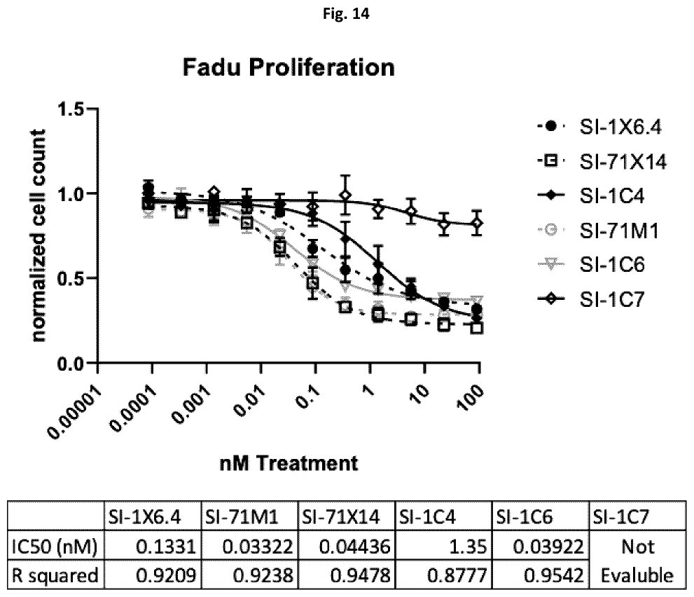

The Fadu response to humanized cetuximab in combination with the HER3 binding domain in bi-specific format (SI-71X14) achieves 3×better anti-proliferative IC-50 compared with the wt cetuximab in the same format (SI-1X6.4). The humanized EGFR in SI-71X14 further achieves a significantly greater overall anti-proliferative effects at higher concentrations (FIG. 13). By way of comparison, SI-1C4 is a bispecific antibody against EGFR and HER3 built on the two-in-one platform described by Schaefer³⁰. IC4 has a similar structure to a monoclonal antibody. The molecule can bind to either EGFR or HER3 on each Fab arm, but cannot engage both targets simultaneously on each Fab arm. While blocking either EGFR or HER3, and in excess, both receptors, the anti-proliferative performance of the two-in-one is inferior to both the bi-specific format of SI-71X14 and SI-1X6.4 (FIG. 14). By way of comparison, SI-1R12 is MM-111, a HER2×HER3 bispecific that has reported anti-proliferative effects. However, while Fadu express both HER2 and HER3, inhibition is not achieved. This attests to the key combination of EGFR facilitation of HER3 blocking antiproliferative effect, rather than HER2 and HER3 on this cell line (FIG. 13).

The reengineering of the cetuximab antibody enable greater anti-proliferative potency when engineered in multi-specific formation compared with wt cetuximab as demonstrated in T cell engager bi and penta specific structures, as well when in combination with HER3 binding domains. The humanized cetuximab domain also achieves greater overall anti-proliferative effects when combined with HER3 binding domains compared to wt cetuximab.

TABLE 1a
The KD value of a TAA binding domain may
vary in different therapeutic antibodies.

| | | | EGFR | HER2 | HER3 | |
|---|---|---|---|---|---|---|
| | | | affinity | affinity | affinity | |
| Therapeutics | Type of | | KD | KD | KD | EC₅₀ |
| Candidate | Mab | Target | (nM) | (nM) | (nM) | (nM) |
| Cetuximab¹¹ | Bivalent | EGFR | 0.2 | | | |
| Panitumumab¹² | Bivalent | EGFR | 0.05 | | | |
| Nimotuzumab¹³ | Bivalent | EGFR | 67 | | | |
| Necitumumab²¹ | Bivalent | EGFR | 0.28 | | | |
| Trastuzumab¹⁴ | Bivalent | HER2 | | 1.8 | | |
| Pertuzumab¹⁵ | Bivalent | HER2 | | 0.8 | | |
| Patritumab¹⁶ | Bivalent | HER3 | | | 1-3 | |
| MM-121¹⁷ | Bivalent | HER3 | | | 0.75 | |
| MM-111¹⁸ | Monovalent | HER2 & | | 0.3 | 16 | |
| | bispecific | HER3 | | | | |
| | HSA-scFv* | | | | | |
| Duligotuzumab | “2-in-1” | EGFR or | 19.9 | | 2.63 | 0.068-0.589 # |
| (MEHD7945A)¹⁹ | Monovalent | HER3 | | | | |
| | bispecific | | | | | |
| SI-1X6.4(C3) | Bivalent | EGFR & | 5.38 | | 107 | |
| | bispecific | HER3 | | | | |

# Results of ADCC analyses using Fadu and NCI-H1975 cells, respectively.

TABLE 1b
The antibodies having therapeutic binding domains
for targeting either EGFR, HER3, or both.

| | | Origin of | | Origin of | |
|---|---|---|---|---|---|
| | | anti-EGFR | EGFR | anti-HER3 | HER3 |
| Protein | Specificity | Fab | valency | scFv | valency |
| SI-1C3 | EGFR | Panitumumab | Bi | n/a | n/a |
| SI-1C5 | EGFR | Nimotuzumab | Bi | n/a | n/a |
| SI-1C6 | EGFR | Cetuximab | Bi | n/a | n/a |
| SI-71M1 | EGFR | Hu-Cetuximab | Bi | n/a | n/a |
| SI-1C7 | HER3 | Fc-scFv | n/a | MM-111 | Bi |
| SI-1X2 | EGFR × | Panitumumab | Bi | MM-111 | Bi |
| | HER3 | | | | |
| SI-1X4.2 | EGFR × | Nimotuzumab | Bi | MM-111 | Bi |
| | HER3 | | | | |
| SI-1X6.4 | EGFR × | Cetuximab | Bi | MM-111 | Bi |
| | HER3 | | | | |
| SI-71X14 | EGFR × | Hu-Cetuximab | Bi | MM-111 | Bi |
| | HER3 | | | | |
| SI-1C4 | EGFR × | Duligotu- | Mono | Duligotu- | Mono |
| | HER3 | zumab | | zumab | |
| SI-1R12 | HER2 × | n/a | n/a | MM-111 | Mono |
| | HER3 | | | | |

TABLE 2
Binding kinetics (affinity) of anti-EGFR proteins
to His-tagged human EGFR extracellular domain,
as measured by biolayer interferometry.

| Protein | KD (M) | kon (1/Ms) | kdis (1/s) |
|---|---|---|---|
| SI-71X14 | 4.61E−09 | 2.64E+05 | 1.22E−03 |
| SI-71M1 | 4.76E−09 | 2.72E+05 | 1.30E−03 |
| SI-1X6.4 | 5.38E−09 | 2.89E+05 | 1.56E−03 |
| SI-1C6 | 5.34E−09 | 2.71E+05 | 1.45E−03 |
| SI-1C3 | 2.28E−09 | 1.95E+05 | 4.45E−04 |
| SI-1X2 | 2.77E−09 | 1.97E+05 | 5.46E−04 |
| SI-1C4 | 1.46E−08 | 3.00E+05 | 4.38E−03 |
| SI-1C5 | 1.58E−08 | 1.13E+05 | 1.78E−03 |
| SI-1X4.2 | 1.88E−08 | 1.08E+05 | 2.02E−03 |

TABLE 3
Binding kinetics (affinity) of anti-HER3 proteins to His-tagged
human HER3 extracellular domain, as measured by biolayer
interferometry. Note that in contrast to all other measurement,
which were determined using AHC sensors, SI-1R12 required
setup with AR2G sensors due to lack of Fc domain.

| Protein | KD (M) | kon (1/Ms) | kdis (1/s) |
|---|---|---|---|
| SI-71X14 | 1.17E−07 | 2.65E+05 | 3.08E−02 |
| SI-1C7 | 1.49E−07 | 2.24E+05 | 3.33E−02 |
| SI-1C4 | 4.29E−09 | 2.05E+05 | 8.77E−04 |
| SI-1X6.4 | 1.07E−07 | 2.82E+05 | 3.02E−02 |
| SI-1X2 | 1.31E−07 | 2.75E+05 | 3.61E−02 |
| SI-1X4.2 | 1.46E−07 | 2.60E+05 | 3.80E−02 |
| SI-1R12 | 9.56E−08 | 4.41E+05 | 4.22E−02 |

TABLE 4
Binding kinetics (avidity) of anti-EGFR proteins
to biotinylated human EGFR extracellular domain,
as measured by biolayer interferometry.

| Protein | KD (M) | kon(1/Ms) | kdis(1/s) |
|---|---|---|---|
| SI-1C7 | N.D. | N.D. | N.D. |
| SI-1X2 | <1.0E−12 | 2.81E+05 | <1.0E−07 |
| SI-1X4.2 | 3.98E−10 | 1.07E+05 | 4.25E−05 |
| SI-1X6.4 | <1.0E−12 | 3.20E+05 | <1.0E−07 |
| SI-71M1 | <1.0E−12 | 4.66E+05 | <1.0E−07 |
| SI-71X14 | <1.0E−12 | 3.53E+05 | <1.0E−07 |

TABLE 5
Binding kinetics (affinity) of anti-EGFR × HER3 proteins and
controls to human His-tagged HER3 following binding to biotinylated
human EGFR in sandwich-type Octet assay. Note that monospecific
anti-EGFR (SI-71M1) and anti-HER3 (SI-1C7) proteins did not show
any binding signal during the HER3 association step, as expected.

| Protein | KD (M) | kon(1/Ms) | kdis(1/s) |
|---|---|---|---|
| SI-1C7 | N.D. | N.D. | N.D. |
| SI-1X2 | 7.70E−07 | 3.45E+04 | 2.65E−02 |
| SI-1X4.2 | 1.65E−07 | 2.79E+05 | 4.59E−02 |
| SI-1X6.4 | 6.17E−07 | 2.09E+05 | 1.29E−01 |
| SI-71M1 | N.D. | N.D. | N.D. |
| SI-71X14 | 9.22E−07 | 7.59E+04 | 7.00E−02 |

TABLE 6
Binding kinetics of anti-HER3 proteins to His-tagged human
EGFR extracellular domain, as measured by biolayer interferometry.
Note that in contrast to all other measurement, which
were determined using AHC sensors, SI-1R12 required setup
with AR2G sensors due to lack of Fc domain.

| Protein | Tm (° C.) |
|---|---|
| SI-71X14 | 64.1 |
| SI-71M1 | 77.8 |
| SI-1C7 | 63.17 |
| SI-1C4 | 69.8 |
| SI-1X6.4 | 62.33 |
| SI-1C6 | 68.39 |
| SI-1R12 | 60.41 |
| SI-1C5 | 65.79 |
| SI-1C3 | 77.05 |
| SI-1X2 | 63.73 |
| SI-1X4.2 | 63.79 |

### SEQUENCE LISTING

### Sequences of αEGFR Variable Domains

| | Amino acid | Nucleotide |
|---|---|---|
| Sequence | seq. ID | seq. ID |
| SI-71M1/SI-71X14 αEGFR VH | 1 | 2 |
| SI-71M1/SI-71X14 αEGFR VL | 3 | 4 |
| SI-71X14 αHER3 VH | 5 | 6 |
| SI-71X14 αHER3 VL | 7 | 8 |

### Sequences of Monoclonal Antibody and Bispecific Antibody

| Sequence | Amino acid seq. ID | Nucleotide seq. ID |
|---|---|---|
| SI-71M1 HC | 9 | 10 |
| SI-71M1 LC | 11 | 12 |
| SI-71X14 HC | 13 | 14 |
| SI-71X14 LC | 11 | 12 |

>Sequence ID 1: SI-71X14 αEGFR VH amino acid sequence
QVOLQQSGPGLVKPSETLSITCTVSGFSLTNYGVHWIRQAPGKGLEWLGVIWSGGNTDYNTPFT
SRFTITKDNSKNQVYFKLRSVRADDTAIYYCARALTYYDYEFAYWGQGTLVTVSS
>Sequence ID 2: SI-71X14 αEGFR VH nucleotide sequence
CAAGTTCAGTTGCAGCAGTCTGGCCCTGGCCTGGTCAAGCCTTCTGAGACACTGTCCATCACCT
GTACCGTGTCCGGCTTCTCCCTGACCAATTACGGCGTGCACTGGATCAGACAGGCCCCTGGCAA
AGGACTGGAATGGCTGGGAGTGATTTGGAGCGGCGGCAACACCGACTACAACACCCCTTTCACC
AGCCGGTTCACCATCACCAAGGACAACTCCAAGAACCAGGTGTACTTCAAGCTGCGGAGCGTGC
GGGCTGATGACACCGCCATCTACTACTGTGCTCGGGCCCTGACCTACTACGACTACGAGTTTGC
TTACTGGGGCCAGGGCACCCTGGTCACAGTTTCTTCT
>Sequence ID 3: SI-71X14 αEGFR VL amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLTVL
>Sequence ID 4: SI-71X14 αEGFR VL nucleotide sequence
GAGATCGTGCTGACCCAGTCTCCTTCCACACTGTCTGTGTCTCCCGGCGAGAGAGCCACCTTCA
GCTGTAGAGCCTCTCAGTCCATCGGCACCAACATCCACTGGTATCAGCAGAAGCCCGGCAAGCC
TCCTCGGCTGCTGATTAAGTACGCCTCCGAGTCCATCAGCGGCATCCCTGACAGATTCTCCGGC
TCTGGCTCTGGCACCGAGTTTACCCTGACCATCTCCTCCGTGCAGTCCGAGGATTTCGCCGTGT
ACTACTGCCAGCAGAACAACAACTGGCCCACCACCTTTGGACCCGGCACCAAGCTGACCGTGCT
G
>Sequence ID 5: SI-71X14 αHER3 VH amino acid sequence
QVQLQESGGGLVKPGGSLRLSCAASGFTFSSYWMSWVRQAPGKGLEWVANINRDGSASYYVDSV
KGRFTISRDDAKNSLYLQMNSLRAEDTAVYYCARDRGVGYFDLWGRGTLVTVSS
>Sequence ID 6: SI-71X14 αHER3 VH nucleotide sequence
CAGGTGCAATTGCAGGAGTCGGGGGGAGGCCTGGTCAAGCCTGGAGGGTCCCTGAGACTCTCCT
GTGCAGCCTCTGGATTCACCTTTAGTAGTTATTGGATGAGCTGGGTCCGCCAGGCTCCAGGGAA
GGGGCTGGAGTGGGTGGCCAACATAAACCGCGATGGAAGTGCGAGTTACTATGTGGACTCTGTG
AAGGGCCGATTCACCATCTCCAGAGACGACGCCAAGAACTCACTGTATCTGCAAATGAACAGCC
TGAGAGCTGAGGACACGGCTGTGTATTACTGTGCGAGAGATCGTGGGGTGGGCTACTTCGATCT
CTGGGGCCGTGGCACCCTGGTCACCGTCTCGAGC
>Sequence ID 7: SI-71X14 αHER3 VL amino acid sequence
QSALTQPASVSGSPGQSITISCTGTSSDVGGYNFVSWYQQHPGKAPKLMIYDVSDRPSGVSDRF
SGSKSGNTASLIISGLQADDEADYYCSSYGSSSTHVIFGGGTKVTVL
>Sequence ID 8: SI-71X14 αHER3 VL nucleotide sequence
CAGTCTGCCCTGACTCAGCCTGCCTCCGTGTCTGGGTCTCCTGGACAGTCGATCACCATCTCCT
GCACTGGAACCAGCAGTGACGTTGGTGGTTATAACTTTGTCTCCTGGTACCAACAACACCCAGG
CAAAGCCCCCAAACTCATGATCTATGATGTCAGTGATCGGCCCTCAGGGGTGTCTGATCGCTTC
TCCGGCTCCAAGTCTGGCAACACGGCCTCCCTGATCATCTCTGGCCTCCAGGCTGACGACGAGG
CTGATTATTACTGCAGCTCATATGGGAGCAGCAGCACTCATGTGATTTTCGGCGGAGGGACCAA
GGTGACCGTCCTA
>Sequence ID 9: SI-71M1 HC amino acid sequence
QVQLQQSGPGLVKPSETLSITCTVSGFSLTNYGVHWIRQAPGKGLEWLGVIWSGGNTDYNTPFT
SRFTITKDNSKNQVYFKLRSVRADDTAIYYCARALTYYDYEFAYWGQGTLVTVSSASTKGPSVF
PLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLSLSPG
>Sequence ID 10: SI-71M1 HC nucleotide sequence
CAAGTTCAGTTGCAGCAGTCTGGCCCTGGCCTGGTCAAGCCTTCTGAGACACTGTCCATCACCT
GTACCGTGTCCGGCTTCTCCCTGACCAATTACGGCGTGCACTGGATCAGACAGGCCCCTGGCAA
AGGACTGGAATGGCTGGGAGTGATTTGGAGCGGCGGCAACACCGACTACAACACCCCTTTCACC
AGCCGGTTCACCATCACCAAGGACAACTCCAAGAACCAGGTGTACTTCAAGCTGCGGAGCGTGC
GGGCTGATGACACCGCCATCTACTACTGTGCTCGGGCCCTGACCTACTACGACTACGAGTTTGC
TTACTGGGGCCAGGGCACCCTGGTCACAGTTTCTTCTGCTAGCACCAAGGGCCCATCGGTCTTC
CCCCTGGCACCCTCCTCCAAGAGCACCTCTGGGGGCACAGCGGCCCTGGGCTGCCTGGTCAAGG
ACTACTTCCCCGAACCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGGTGACCGTGCCCTCC
AGCAGCTTGGGCACCCAGACCTACATCTGCAACGTGAATCACAAGCCCAGCAACACCAAGGTGG
ACAAGAGAGTTGAGCCCAAATCTTGTGACAAAACTCACACATGCCCACCGTGCCCAGCACCTGA
ACTCCTGGGGGGACCGTCAGTCTTCCTCTTCCCCCCAAAACCCAAGGACACCCTCATGATCTCC
CGGACCCCTGAGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGCTGAATGGCAAGGAG
TACAAGTGCAAGGTCTCCAACAAAGCCCTCCCAGCCCCCATCGAGAAAACCATCTCCAAAGCCA
AAGGGCAGCCCCGAGAACCACAGGTGTACACCCTGCCCCCATCCCGGGATGAGCTGACCAAGAA
CCAGGTCAGCCTGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCTCCGGGT
TAG
>Sequence ID 11: SI-71M1 and SI-71X14 LC amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLTVLRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC
>Sequence ID 12: SI-71M1 and SI-71X14 LC nucleotide sequence
GAGATCGTGCTGACCCAGTCTCCTTCCACACTGTCTGTGTCTCCCGGCGAGAGAGCCACCTTCA
GCTGTAGAGCCTCTCAGTCCATCGGCACCAACATCCACTGGTATCAGCAGAAGCCCGGCAAGCC
TCCTCGGCTGCTGATTAAGTACGCCTCCGAGTCCATCAGCGGCATCCCTGACAGATTCTCCGGC
TCTGGCTCTGGCACCGAGTTTACCCTGACCATCTCCTCCGTGCAGTCCGAGGATTTCGCCGTGT
ACTACTGCCAGCAGAACAACAACTGGCCCACCACCTTTGGACCCGGCACCAAGCTGACCGTGCT
GCGTACGGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCAACAGGGGAGAGT
GTTAG
>Sequence ID 13: SI-71X14 HC amino acid sequence
QVQLQQSGPGLVKPSETLSITCTVSGFSLTNYGVHWIRQAPGKGLEWLGVIWSGGNTDYNTPFT
SRFTITKDNSKNQVYFKLRSVRADDTAIYYCARALTYYDYEFAYWGQGTLVTVSSASTKGPSVF
PLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLSLSPG
GGGGSGGGGSQVQLQESGGGLVKPGGSLRLSCAASGFTFSSYWMSWVRQAPGKGLEWVANINRD
GSASYYVDSVKGRFTISRDDAKNSLYLQMNSLRAEDTAVYYCARDRGVGYFDLWGRGTLVTVSS
GGGGSGGGGSGGGGSQSALTQPASVSGSPGQSITISCTGTSSDVGGYNFVSWYQQHPGKAPKLM
IYDVSDRPSGVSDRFSGSKSGNTASLIISGLQADDEADYYCSSYGSSSTHVIFGGGTKVTVL
>Sequence ID 14: SI-71X14 HC nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCTACTACGACTACGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTGGCGGTGGAGGGTCCGGCGGTGGTGGATCACAGGTGCAATTGCAGGAGTCGGGGGG
AGGCCTGGTCAAGCCTGGAGGGTCCCTGAGACTCTCCTGTGCAGCCTCTGGATTCACCTTTAGTAGTTAT
TGGATGAGCTGGGTCCGCCAGGCTCCAGGGAAGGGGCTGGAGTGGGTGGCCAACATAAACCGCGATGGAA
GTGCGAGTTACTATGTGGACTCTGTGAAGGGCCGATTCACCATCTCCAGAGACGACGCCAAGAACTCACT
GTATCTGCAAATGAACAGCCTGAGAGCTGAGGACACGGCTGTGTATTACTGTGCGAGAGATCGTGGGGTG
GGCTACTTCGATCTCTGGGGCCGTGGCACCCTGGTCACCGTCTCGAGCGGTGGAGGCGGTTCAGGCGGAG
GTGGTTCCGGCGGTGGCGGCTCCCAGTCTGCCCTGACTCAGCCTGCCTCCGTGTCTGGGTCTCCTGGACA
GTCGATCACCATCTCCTGCACTGGAACCAGCAGTGACGTTGGTGGTTATAACTTTGTCTCCTGGTACCAA
CAACACCCAGGCAAAGCCCCCAAACTCATGATCTATGATGTCAGTGATCGGCCCTCAGGGGTGTCTGATC
GCTTCTCCGGCTCCAAGTCTGGCAACACGGCCTCCCTGATCATCTCTGGCCTCCAGGCTGACGACGAGGC
TGATTATTACTGCAGCTCATATGGGAGCAGCAGCACTCATGTGATTTTCGGCGGAGGGACCAAGGTGACC
GTCCTATAA

### REFERENCE

1. Diaz-Serrano, A. et al. Genomic Profiling of HER2-Positive Gastric Cancer: PI3K/Akt/mTOR Pathway as Predictor of Outcomes in HER2-Positive Advanced Gastric Cancer Treated with Trastuzumab. Oncologist.23, 1092-1102 (2018).
2. Durkee, B Y, et al. Cost-Effectiveness of Pertuzumab in Human Epidermal Growth Factor Receptor 2-Positive Metastatic Breast Cancer. Journal of Clinical Oncology. 2016, 34 (9): 902-9.
3. Robinson, Matthew K., et al. “Targeting ErbB2 and ErbB3 with a bispecific single-chain Fv enhances targeting selectivity and induces a therapeutic effect in vitro.” British journal of cancer 99.9 (2008): 1415-1425.
4. Goel, S. & Winer, E. P. POINT: HER2-Targeted Combinations in Advanced HER2-Positive Breast Cancer. Oncology (Williston Park). 29, 797-798, 802 (2015).
5. Luque-Cabal, M. et al. Mechanisms Behind the Resistance to Trastuzumab in HER2-Amplified Breast Cancer and Strategies to Overcome It. Clin. Med. Insights Oncol. 10, 21-30 (2016).
6. Hwang W Y K, Foote J. Immunogenicity of engineered antibodies. Methods 2005; 36:3-10.
7. Park S S, Kim J, Brandts J F, Hong H J. Stability of murine, chimeric and humanized antibodies against pre-S2 surface antigen of hepatitis B virus. Biologicals 2003; 31:295-302.
8. Ayoub D, Jabs W, Resemann A, Evers W, Evans C, Main L, Baessmann C, Wagner-Rousset E, Suckau D, Beck A. Correct primary structure assessment and extensive glyco-profiling of cetuximab by a combination of intact, middle-up, middle-down and bottom-up ESI and MALDI mass spectrometry techniques. MAbs 2013; 5:699-710.
9. van de Bovenkamp F S, Hafkenscheid L, Rispens T, Rombouts Y. The Emerging Importance of IgG Fab Glycosylation in Immunity. J Immunol 2016; 196:1435-41.
10. Chung C H, Mirakhur B, Chan E, Le Q-T, Berlin J, Morse M, Murphy B A, Satinover S M, Hosen J, Mauro D, et al. Cetuximab-Induced Anaphylaxis and IgE Specific for Galactose-α-1,3-Galactose. N Engl J Med 2008; 358:1109-17.
11. Cetuximab: https://www.ema.europa.eu/en/documents/scientific-discussion/ebitux-epar-scientific-discussion_en.pdf
12. Garcia-Foncillas, Jesús, et al. “Distinguishing features of cetuximab and panitumumab in colorectal cancer and other solid tumors.” Frontiers in oncology 9 (2019): 849.
13. Tundidor, Yaima, et al. “Affinity-matured variants derived from nimotuzumab keep the original fine specificity and exhibit superior biological activity.” Scientific reports 10.1 (2020): 1-14.
14. Lakayan, Dina, et al. “Affinity profiling of monoclonal antibody and antibody-drug-conjugate preparations by coupled liquid chromatography-surface plasmon resonance biosensing.” Analytical and bioanalytical chemistry 410.30 (2018): 7837-7848.
15. Pertuzumab: https://www.tga.gov.au/sites/default/files/auspar-pertuzumab-131001.pdf
16. Malm, Magdalena, et al. “Targeting HER3 using mono- and bispecific antibodies or alternative scaffolds.” MAbs. Vol. 8. No. 7. Taylor & Francis, 2016.
17. Aurisicchio, Luigi, et al. “The promise of anti-ErbB3 monoclonals as new cancer therapeutics.” Oncotarget 3.8 (2012): 744.
18. McDonagh, Charlotte F., et al. “Antitumor activity of a novel bispecific antibody that targets the ErbB2/ErbB3 oncogenic unit and inhibits heregulin-induced activation of ErbB3.” Molecular cancer therapeutics 11.3 (2012): 582-593.
19. Schaefer, Gabriele, et al. “A two-in-one antibody against HER3 and EGFR has superior inhibitory activity compared with monospecific antibodies.” Cancer cell 20.4 (2011): 472-486.
20. SI-1X6.4(C3): U.S. Ser. No. 15/119,694.
21. Liu, Meilin, et al. “Identification and characterization of a fully human antibody directed against epidermal growth factor receptor for cancer therapy.” (2004): 163-163.
22. Borras, Leo, et al. “Generic approach for the generation of stable humanized single-chain Fv fragments from rabbit monoclonal antibodies.” Journal of Biological Chemistry 285.12 (2010): 9054-9066.
23. Ewert, Stefan, et al. “Biophysical properties of human antibody variable domains.” Journal of molecular biology 325.3 (2003): 531-553.
24. Nieba, Lars, et al. “Disrupting the hydrophobic patches at the antibody variable/constant domain interface: improved in vivo folding and physical characterization of an engineered scFv fragment.” Protein engineering 10.4 (1997): 435-444.
25. Arteaga, Carlos L., et al. “Treatment of HER2-positive breast cancer: current status and future perspectives.” Nature reviews Clinical oncology 9.1 (2012): 16-32.
26. Price, Katharine A R, and Ezra E. Cohen. “Current treatment options for metastatic head and neck cancer.” Current treatment options in oncology 13.1 (2012): 35-46.
27. Bode, Udo, et al. “Nimotuzumab treatment of malignant gliomas.” Expert opinion on biological therapy 12.12 (2012): 1649-1659.
28. Price, Timothy J., et al. “Panitumumab versus cetuximab in patients with chemotherapy-refractory wild-type KRAS exon 2 metastatic colorectal cancer (ASPECCT): a randomised, multicentre, open-label, non-inferiority phase 3 study.” The Lancet Oncology 15.6 (2014): 569-579.
29. Cohen, Roger B. “Current challenges and clinical investigations of epidermal growth factor receptor (EGFR)- and ErbB family-targeted agents in the treatment of head and neck squamous cell carcinoma (HNSCC).” Cancer treatment reviews 40.4 (2014): 567-577.
30. Schaefer, Gabriele, et al. “A two-in-one antibody against HER3 and EGFR has superior inhibitory activity compared with monospecific antibodies.” Cancer cell 20.4 (2011): 472-486.
31. Geuijen, Cecile A W, et al. “Unbiased combinatorial screening identifies a bispecific IgG1 that potently inhibits HER3 signaling via HER2-guided ligand blockade.” Cancer Cell 33.5 (2018): 922-936.
32. Yu, Shengnan, et al. “A novel asymmetrical anti-HER2/CD3 bispecific antibody exhibits potent cytotoxicity for HER2-positive tumor cells.” Journal of Experimental & Clinical Cancer Research 38.1 (2019): 1-16.
33. Beiboer, Sigrid H W, et al. “Guided selection of a pan carcinoma specific antibody reveals similar binding characteristics yet structural divergence between the original murine antibody and its human equivalent.” Journal of molecular biology 296.3 (2000): 833-849.
34. Khaw, Ban An, et al. “Technetium-99m labeling of antibodies to cardiac myosin Fab and to human fibrinogen.” Journal of Nuclear Medicine 23.11 (1982): 1011-1019.
35. Rousseaux, J., R. Rousseaux-Prevost, and Hervé Bazin. “[63] Optimal conditions for the preparation of proteolytic fragments from monoclonal IgG of different rat IgG subclasses.” Methods in enzymology 121 (1986): 663-669.
36. Köhler, Georges, and Cesar Milstein. “Continuous cultures of fused cells secreting antibody of predefined specificity.” nature 256.5517 (1975): 495-497.
37. U.S. Pat. No. 4,816,567
38. Siegel, D. L. “Recombinant monoclonal antibody technology.” Transfusion clinique et biologique 9.1 (2002): 15-22.
39. Tiller, Thomas. “Single B cell antibody technologies.” New biotechnology 28.5 (2011): 453-457.
40. Seeber, Stefan, et al. “A robust high throughput platform to generate functional recombinant monoclonal antibodies using rabbit B cells from peripheral blood.” PloS one 9.2 (2014): e86184.
41. U.S. Pat. No. 4,816,567
42. Morrison, Sherie L., et al. “Chimeric human antibody molecules: mouse antigen-binding domains with human constant region domains.” Proceedings of the National Academy of Sciences 81.21 (1984): 6851-6855.
43. Queen, Cary, et al. “A humanized antibody that binds to the interleukin 2 receptor.” Proceedings of the National Academy of Sciences 86.24 (1989): 10029-10033.
44. Hodgson, John. “Making monoclonals in microbes.” Bio/technology 9.5 (1991): 421-425.

What is claimed is:

1. A bispecific tetravalent antibody having a binding specificity to a human EGFR (epithelium growth factor receptor), comprising, from N terminus to C terminus,
a Fab region having a first binding specificity to human EGFR, wherein the Fab region comprises a variable region having an amino acid sequence of SEQ ID NO: 1 or 3;
a Fc domain, and
a scFv domain having a second binding specificity to HER3.

2. The bispecific tetravalent antibody of claim 1, comprising an amino acid sequence of SEQ ID NO: 11 or 13.

3-4. (canceled)

5. The bispecific tetravalent antibody of claim 1, wherein the first binding affinity has a KD less than 20 nM, and the second binding affinity has a KD more than about 50 nM.

6. The bispecific tetravalent antibody of claim 1, wherein the Fab region is stapled with a disulphide bond.

7. The bispecific tetravalent antibody of claim 1, wherein the tetravalent bispecific antibody is an isolated monoclonal antibody, a humanized antibody, a chimeric antibody, or a recombinant antibody.

8-9. (canceled)

10. A heavy chain, comprising an amino acid sequence of SEQ ID NO: 9 or 13.

11. A light chain, comprising an amino acid sequence of SEQ ID NO: 11.

12. An isolated nucleic acid encoding the tetravalent bispecific antibody of claim 1.

13. An expression vector comprising the isolated nucleic acid of claim 12.

14. A host cell comprising the nucleic acid of claim 12.

15. A method of producing a tetravalent bispecific antibody, comprising culturing the host cell of claim 14 so that the tetravalent bispecific antibody is produced.

16. A pharmaceutical composition, comprising the tetravalent bispecific antibody of claim 1 and a pharmaceutically acceptable carrier.

17. The pharmaceutical composition of claim 16, further comprising radioisotope, radionuclide, a toxin, a therapeutic agent, a chemotherapeutic agent or a combination thereof.

18. An immunoconjugate comprising the tetravalent bispecific antibody of claim 1 and a cytotoxic agent, wherein the cytotoxic agent comprises a chemotherapeutic agent, a growth inhibitory agent, a toxin, or a radioactive isotope.

22. (canceled)

23. A pharmaceutical composition, comprising the immunoconjugate of claim 18 and a pharmaceutically acceptable carrier.

24. A method of treating a subject with a cancer, comprising administering to the subject an effective amount of the tetravalent bispecific antibody of claim 1

25. The method of claim 24, wherein the cancer comprises cells expressing HER3 or EGFR, and wherein the cancer comprises breast cancer, colorectal cancer, pancreatic cancer, head and neck cancer, melanoma, ovarian cancer, prostate cancer, non-small lung cell cancer, small cell lung cancer, glioma, esophageal cancer, nasopharyngeal cancer, kidney cancer, gastric cancer, liver cancer, bladder cancer, cervical cancer, brain cancer, lymphoma, leukaemia, or myeloma.

26. (canceled)

27. The method of claim 24, further comprising co-administering an effective amount of a therapeutic agent, wherein the therapeutic agent comprises capecitabine, cisplatin, trastuzurnab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, a derivative or a combination thereof.

28-31. (canceled)


SEQUENCE LISTING (14 sequences)

SEQ ID NO: 1 - protein, 119 aa, Artificial Sequence
  Synthesized
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSS

SEQ ID NO: 2 - DNA, 357 bp, Artificial Sequence
  Synthesized
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttct

SEQ ID NO: 3 - protein, 107 aa, Artificial Sequence
  Synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 4 - DNA, 321 bp, Artificial Sequence
  Synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgaccgtgctg

SEQ ID NO: 5 - protein, 118 aa, Artificial Sequence
  Synthesized
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 6 - DNA, 354 bp, Artificial Sequence
  Synthesized
caggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagc

SEQ ID NO: 7 - protein, 111 aa, Artificial Sequence
  Synthesized
QSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 8 - DNA, 333 bp, Artificial Sequence
  Synthesized
cagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtccta

SEQ ID NO: 9 - protein, 448 aa, Artificial Sequence
  Synthesized
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - DNA, 1347 bp, Artificial Sequence
  Synthesized
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 11 - protein, 214 aa, Artificial Sequence
  Synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 645 bp, Artificial Sequence
  Synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgaccgtgctgcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 13 - protein, 702 aa, Artificial Sequence
  Synthesized
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 14 - DNA, 2109 bp, Artificial Sequence
  Synthesized
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa